SEQUENCE LISTING (2 sequences)

SEQ ID NO: 1 - DNA, 39 bp, Artificial Sequence
  Description of Artificial Sequence Synthetic oligonucleotide
cacaggctacggcacgtagagcatcaccatgatcctgtg

SEQ ID NO: 2 - DNA, 50 bp, Artificial Sequence
  Description of Artificial Sequence Synthetic oligonucleotide
tttttttttatctaactgctgcgccgccgggaaaatactgtacggttaga

(19) United States
(12) Patent Application Publication
(10) Pub. No.: US 2014/0296095 A1
(43) Pub. Date: Oct. 2, 2014

(54) Spatially Selective Release of Aptamer-Captured Cells by Temperature Mediation

(71) Applicant: The Trustees of Columbia University in the City of New York, New York, NY (US)

(72) Inventors: Qiao Lin, New York, NY (US); Jing Zhu, New York, NY (US); Milan N. Stojanovic, Fort Lee, NY (US); Renjun Pei, Suzhou (CN)

(73) Assignee: The Trustees of Columbia University in the City of New York, New York, NY (US)

(21) Appl. No.: 14/223,767

(22) Filed: Mar. 24, 2014

Related U.S. Application Data
(63) Continuation-in-part of application No. PCT/US12/56926, filed on Sep. 24, 2012.
(60) Provisional application No. 61/931,389, filed on Jan. 24, 2014.
(60) Provisional application No. 61/674,183, filed on Jul. 20, 2012.
(60) Provisional application No. 61/538,768, filed on Sep. 23, 2011.

(51) Int. Cl. G01N 33/574

(52) U.S. Cl. CPC G01N 33/57492; G01N 33/57426
USPC 506/9; 435/6.14; 435/289.1; 506/16; 435/286.1

(57) ABSTRACT

Methods and systems are provided for capturing and releasing target cells. The system includes a microdevice having a microchamber including surface-patterned aptamers capable of binding with the target cells. A sample including target cells is introduced to the microchamber, where the target cells bind to the aptamers at locally regulated temperatures. The captured target cells can be selectively released when the temperature of a region is changed to a second temperature.

### CROSS REFERENCES TO RELATED APPLICATIONS

This application is a continuation-in-part of PCT/US12/056,926, filed Sep. 24, 2012, and which claims priority from U.S. Provisional Application No. 61/538,768, filed Sep. 23, 2011, Provisional Application No. 61/674,183, filed Jul. 20, 2012, and Provisional Application No. 61/931,389, filed Jan. 24, 2014, the disclosure of each of which is incorporated herein in its entirety.

### STATEMENT REGARDING FEDERALLY FUNDED RESEARCH

This invention was made with government support under CBET-0854030, awarded by the National Science Foundation; RR025816-02 and CA147925-01, both awarded by the National Institutes of Health. The government has certain rights in this invention.

### BACKGROUND

Specific cell isolation is important in basic biological research and clinical diagnostics. Antibodies that are specific to cell membrane proteins are most often employed to achieve this goal. For example, magnetic-activated cell sorting (MACS) and fluorescence-activated cell sorting (FACS) are highly attractive because of their high specificity to target cells. The MACS method uses the presence or absence of magnetic forces to recognize different cell types. Although it is amenable to high-throughput operations, there is generally no difference between the magnetic forces generated by microbeads with different surface-modified antibodies specific to different target cells. Therefore, MACS is a single-parameter cell isolation method, and can lack the capability to distinguish and sort multiple types of cells. FACS uses different species of antibodies with different fluorescent labels to recognize target cells. Multiple characteristics of cells can be monitored, and thus different cell types can be separated and collected simultaneously. However, the application of FACS can be restricted by its relatively low yield and complex and expensive experimental instrumentation.

Microfluidic technologies can enable more efficient and effective cell isolation with improved sensitivity and resolution, minimized sample and reagent consumption, lower cost and the capability of automation and point-of-care. To achieve specific cell isolation, antibodies are employed. For example, the isolation of rare circulating tumor cells from whole blood samples has been achieved in a microfluidic device with micropillars that are functionalized with anti-epithelial cell adhesion molecule antibodies. Unfortunately, antibodies are not always stable, and are expensive and time-consuming to develop. In addition, in order to achieve molecular and functional analysis or cell-based therapeutics, cells can be released with minimal contamination and negligible disruption to their viability. However, the interaction between antibodies and antigens are not necessarily reversible under normal physiological conditions. Cells are hence typically released from antibody-functionalized surfaces using trypsin to digest antibody-specific cell membrane proteins, or varying the substrate hydrophobicity to detach hydrophobically anchored antibodies. Tryptic digestion is not efficient, only applicable to a small portion of biomarkers involved in affinity cell capture, and can influence cell viability and phenotypic properties. Meanwhile, temperature dependent substrate property alteration cannot cause the dissociation of antibodies from the antigens, leaving the antibodies attached to the cell membranes. Therefore there is a need for methods that allow rapid and non-destructive release of cells from affinity surfaces.

Aptamers, which are oligonucleotides that bind specifically to target molecules, can be selected from a randomized oligonucleotide library using a synthetic process. Compared with antibodies, aptamers are stable, designable and amenable to chemical modifications. Meanwhile, the binding between aptamers and target molecules is reversible because of conformational changes caused by temperature variations. In addition, aptamers for multiple cellular targets, such as acute lymphoblastic leukemia (ALL) precursor T cells, liver cancer cells and stem cells, are available. These aptamers bind to cell membrane proteins by hydrogen bonds, hydrophobic interactions, van der Waals interactions, aromatic stacking or their combinations. Such affinity binding allows the aptamers to capture target cells specifically.

For example, aptamers targeting prostate-specific membrane antigen have been used in a microfluidic system to separate LNCaP cells from a heterogeneous cell mixture. Release of aptamer-captured cells has been accomplished by methods such as exonuclease degradation of aptamers [27], air bubble dislodging and temperature stimulation. Unfortunately, the use of exonuclease is inefficient because of the slow diffusive transport of enzymes and the low enzymatic reaction rate, whereas the use of air bubbles can damage cells and generate dead volumes leading to low cell release efficiency.

There is, therefore, a need for microfluidic methods and systems for selective capture and efficient, nondestructive release of cells for detection and diagnostics.

### SUMMARY

The disclosed subject matter provides techniques for capturing and releasing target cells. Methods and systems are provided for an aptamer-based microfluidic device with a surface selectively functionalized with cell-specific aptamers and integrated microheaters with temperature sensors to achieve specific cell capture and temperature-mediated release of selected groups of cells. Aptamers can be patterned on design-specified regions of the chip surface, and the heat generated by the microheaters can be restricted to each aptamer-functionalized chip area. Target cells can be captured by the surface-patterned aptamers with high specificity. A temperature change can be produced using one group of microheater and temperature sensor to reversibly break cell-aptamer binding in the selected chip area, allowing the release and retrieval of viable target cells from this region for downstream applications. After the temperature change is reversed, the aptamer-functionalized surface can recover its binding affinity to target cells. In one experiment, the disclosed methods and systems were applied to CCRF-CEM cells, a human ALL T cell line and sgc8c, an aptamer specific to these cells, to demonstrate its capability for specific capture and non-destructive, spatially selective temperature-mediated release of target cells.

In an exemplary method for capturing and releasing target cells, a sample is introduced into the microchamber, so that the target sells bind to the aptamer at an initial temperature, and are released at a second, different temperature. The sample can include impurities other than the target cells, such as non-target cells, small molecules, and proteins. Before releasing the aptamer-bound target cells, the microchamber can be washed to remove cells or other substances not bound to the aptamer. After the washing, a second sample including the target cells can be introduced into the microchamber to increase the amount of the target cells to bind with the aptamer.

In some embodiments, the target cells have membrane protein(s) and the binding between the target cells and the aptamer are via the interaction between the aptamer and the membrane protein(s). The aptamer can be selected or developed to specifically bind with the membrane protein. In one embodiment, the membrane protein is PTK7.

In some embodiments, the initial temperature can be about 20° C. to 30° C., e.g., at about room temperature. In alternative embodiments, the initial temperature can be at physiological temperature (about 37° C.). In some embodiments, the second temperature for the release of the target cells can be about from 35° C. to about 55° C., e.g., at about 48° C. In alternative embodiments, the second temperature can be lower than the initial temperature, e.g., the second temperature can be from about 4° C. to about 37° C.

### BRIEF DESCRIPTION OF THE DRAWINGS

FIGS. 1a-1c are schematic diagrams illustrating cell capture and temperature-mediated release according to some embodiments of the disclosed subject matter.

FIG. 2a is a schematic diagram of a microdevice for selective capture and release of target cells according to some embodiments of the disclosed subject matter. All dimensions are in microns.

FIGS. 2b-2g are schematic diagrams of an example fabrication procedure of the microdevice depicted in FIG. 2a.

FIG. 2h is an image of an example microdevice according to some embodiments of the disclosed subject matter.

FIG. 2i is a close-up image of a portion of the image depicted in FIG. 2h.

FIG. 2j is an example setup for operating a microdevice for capturing and temperature-mediated release of target cells according to some embodiments of the disclosed subject matter.

FIGS. 3a-3d present results of certain tests performed according to some embodiments of the disclosed subject matter. FIGS. 3a and 3b are images of the microchamber of a microdevice after the introduction of a sample, and after the introduction of 10 samples and buffer washing, respectively. FIG. 3c is a plot depicting the time response of the amount of captured cells versus incubation time. FIG. 3d is a plot depicting the concentration response of cell capture.

FIG. 4a-4e present results of certain tests performed according to some embodiments of the disclosed subject matter. FIG. 4a is a plot depicting percentage of captured cells remaining on the substrate as a function of time while rinsing at constant temperature (48° C. and room temperature) and flow rate (5 μL/min); FIG. 4b is a plot depicting captured cell density versus the number of cell suspension samples introduced while the temperature was maintained at either 48° C. or room temperature; FIG. 4c is a plot showing the effect of temperature on cell release efficiency while rinsing at 5 μL/min; FIG. 4d is a plot showing the effect of flow rate on cell release efficiency while the microchamber temperature was maintained at 48° C.; FIG. 4e is a bar graph showing cell capture and re-capture on the regenerated aptamer-functionalized surface: the normalized percentage of remaining cells after the first, second and third capture and regeneration cycle.

FIGS. 5a-5c illustrate the viability of cells subjected to capture and release according to some embodiments of the disclosed subject matter. FIGS. 5a and 5b are image of PI stained cells (in 5a) and JC-1 stained cells (in 5b) following cell capture and release, generated by a combination of phase contrast and fluorescent micrographs. FIG. 5c is a bar graph showing concentrations of normal cells and heat-treated cells as a function of culture duration.

FIGS. 6a-6c are schematic diagrams illustrating specific cell capture and spatially selective temperature-mediated cell release according to some embodiments of the disclosed subject matter. FIG. 6a illustrates cell capture at room temperature. FIG. 6b illustrates D-PBS wash to remove non-target cells. FIG. 6c illustrates temperature-mediated release of a selected group of cell.

FIG. 7 is a schematic diagram of a microfluidic device for specific cell capture and spatially selective temperature-mediated cell release according to some embodiments of the disclosed subject matter.

FIGS. 8a-8d present schematic diagrams of microchip fabrication and aptamer immobilization according to some embodiments of the disclosed subject matter. FIG. 8a shows deposition, patterning and passivation of gold heaters. FIG. 8b shows deposition, patterning and passivation of gold heater sensors. FIG. 8c shows attachment of a PDMS membrane with through holes onto the microchip, and functionalization of biotinylated aptamers. FIG. 8d shows removal of the PDMS membrane.

FIG. 9a-9d illustrate microchamber fabrication and bonding according to some embodiments of the disclosed subject matter. FIG. 9a shows fabrication of SU-8 mold. FIG. 9b shows casting of a PDMS microchamber. FIG. 9c shows bonding of the PDMS microchamber onto the microchip. FIG. 9d is a photograph of a fabricated microfluidic device, and micrograph of the microheaters and sensors.

FIG. 10 illustrates the experimental setup for specific cell capture and spatially selective temperature-mediated cell release according to one embodiment of the disclosed subject matter.

FIG. 11 illustrates immobilization of aptamers in design-specified regions of the chip surface according to one embodiment of the disclosed subject matter.

FIGS. 12a-12d illustrate specific cell capture and spatially selective temperature-mediated cell release according to some embodiments of the disclosed subject matter. FIG. 12a shows CCRF-CEM cells captured by the aptamer functionalized surface. FIG. 12b shows temperature-mediated cell release in regions 2 and 3. FIG. 12c shows temperature-mediated cell release in region 4. FIG. 12d shows specific cell recapture on the same aptamer functionalized surface.

FIG. 13 is a micrograph of JC-1 stained cells following cell capture and temperature-mediated cell release performed on a microfluidic device.

### DETAILED DESCRIPTION

The disclosed subject matter provides techniques for selective capture and release of target cells. A device incorporating a microchamber can be provided, including an aptamer capable of binding with the target cells. A sample including target cells can be introduced to the microchamber so that the target sells bind to the aptamer at an initial temperature, and are released at a second, different temperature.

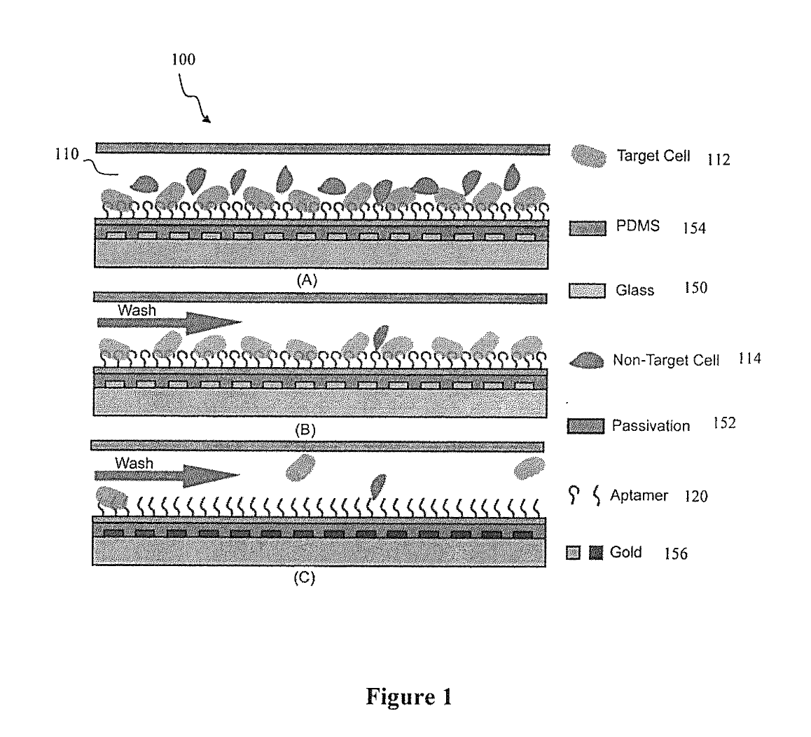

In example embodiments, as illustrated in FIG. 1, the techniques utilize a microdevice 100 including a microchamber (or chamber) 110 which is functionalized on its inner surface with aptamers 120 that bind with the target cells 112, e.g., via certain membrane proteins of the target cells. The dimensions noted on FIG. 1 are only for purpose of illustration. The overall length and width of the microdevice can be a few millimeters. The length and width of the microchamber can be in the order of millimeters, and the depth of the microchamber can be from a few microns to a tens of microns to allow transportation of the target cells while retaining reasonable encounter probability between cells and aptamer. For example, the depth of the chamber can be from about 10 to about 100 microns. When a sample including the target cells is introduced to the microchamber, the aptamers bind with the target cells at a first temperature, e.g., room temperature (FIG. 1a). Thereafter, the microchamber can be washed to remove impurities in the sample, e.g., non-target cells, small molecules, proteins, or the like, that are not bound with the aptamers (FIG. 1b). For certain target cells, the cell capture procedure can also be conducted at physiological condition (about 37° C.).

The aptamer can be immobilized on an inner surface of the microchamber by various techniques available to those skilled in the art, such as physical interactions or chemical bonding. For example, the inner surface of the microchamber can be functionalized by certain proteins, e.g., streptavidin, which can bind an aptamer tagged with biotin. Alternatively, the inner surface of the microchamber can be modified with functional groups, e.g., a thiol group. The thiol group can then be connected by crosslinker, e.g. N-gamma-Maleimidobutyryl-oxysuccinimide ester, together with the streptavidin.

If desired, another sample including the target cells can be introduced into the microchamber to allow increased amount of target cells to bind with the aptamers. To release the captured target cells from the aptamers, the temperature of the microchamber can be raised, e.g., via integrated resistive heaters 156 on the microchip, to a second, higher temperature to disrupt the binding between the aptamer and the target cells while maintaining the structural integrity and viability of the cells (FIG. 1c). The released cells can be collected for further analysis or detection. The microdevice and the aptamers can be reused for processing further samples. In alternative embodiments, the cell release can be achieved at a temperature lower than the initial temperature, e.g., by cooling the microchamber to disrupt the interactions between the aptamer and the bound target cells. Such cooling can be thermoelectric cooling, e.g., by using a Peltier element incorporated as a part of the microdevice. For example, a suitable aptamer for MUC1 cells can capture MUC1 cells at about physiological condition (about 37° C.) and release the cells at a lower temperature, e.g., about 4° C., or at a temperature higher than 37° C.

The microdevice used in the above-described procedure can be fabricated using standard microfabrication techniques, as will be further described in Example 1. Briefly, as shown in FIGS. 1a-1c, the microchamber 110 can be formed between a cavity or void between two PDMS layers 154, the bottom layer positioned atop a passivation layer 152 that covers embedded heaters 156, which are deposited on glass substrate 150.

The target cells can be any cells that have surface membrane proteins to which an aptamer can be selected or developed to specifically bind. For example, the cells can include CCRF-CEM, MCF7, LNCaP, Hs578T, and the corresponding membrane proteins can include PTK7, MUC1, PSMA, and PDGF, respectively.

The aptamers can be selected based on the membrane proteins of the target cells, or developed using SELEX procedure based on membrane proteins of the target cells. Particular aptamers can be generated which bind with specific equilibrium constants, kinetic parameters, and at specific temperatures. For example, for CCRF-CEM cells, a suitable aptamer can be sgc8c. For MCF-7 cells, a suitable aptamer can be MUC1-5TR-1. Aptamers for PSMA (on LNCaP cells) and PDGF (on Hs578T cell line) can be xPSM-A9 and PDGF-aptamer-36t, respectively. PDGF-aptamer-36t has a sequence of: 5′-CAC AGG CTA CGG CAC GTA GAG CAT CAC CAT GAT CCT GTG-3′ (SEQ ID NO:1).

The first temperature at which the aptamer binds with the target cells depend on the choice of aptamer-membrane protein of the target cells. In example embodiments, the first temperature can be about from 20° C. to about 30° C., e.g., about 25° C. In other example embodiments, the first temperature can be about 37° C. Likewise, the second temperature at which the captured cells are released from the aptamer can also depend on the choice of aptamer-membrane protein of the target cells. In example embodiments, the second temperature can be about from 30° C. to about 55° C., e.g., about 48° C. In alternative embodiments, the second temperature can be from about 4° C. to about 37° C. The duration of heating or cooling at the second temperature can be brief, e.g., between 1 to 5 minutes, e.g., about 2 minutes.

Further details of device structure, fabrication, and operation procedures of the above-described embodiments can be found in the following Examples, which are provided for illustration purpose only and not for limitation.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

### Example 1

This Example describes the fabrication of an example microdevice as well as capture and temperature-mediated release of target cells using the microdevice and CCRF-CEM cells for illustration. CCRF-CEM cells are a human ALL cell line. ALL is a common cancer for children younger than 14 years old, representing one third of all malignancies in that age group. CCRF-CEM cells can be recognized by the DNA aptamer sgc8c. Toledo cells, a human diffuse large-cell lymphoma cell line not recognized by sgc8c, were used as a control (non-target cells).

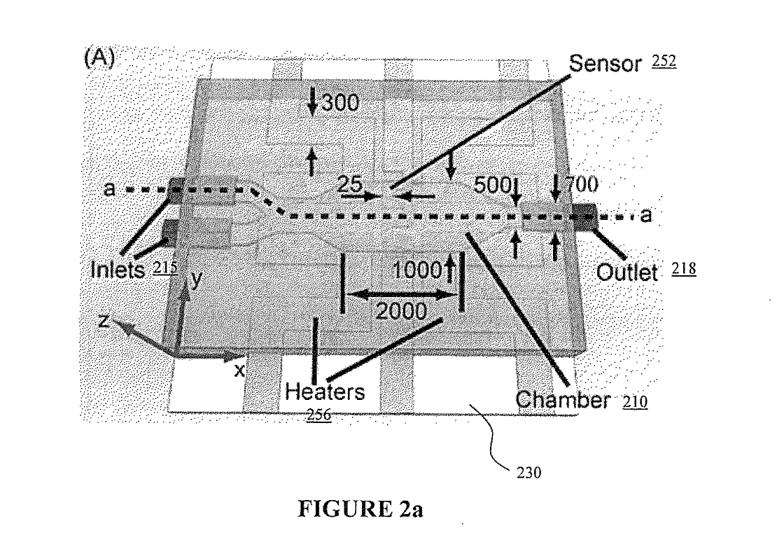

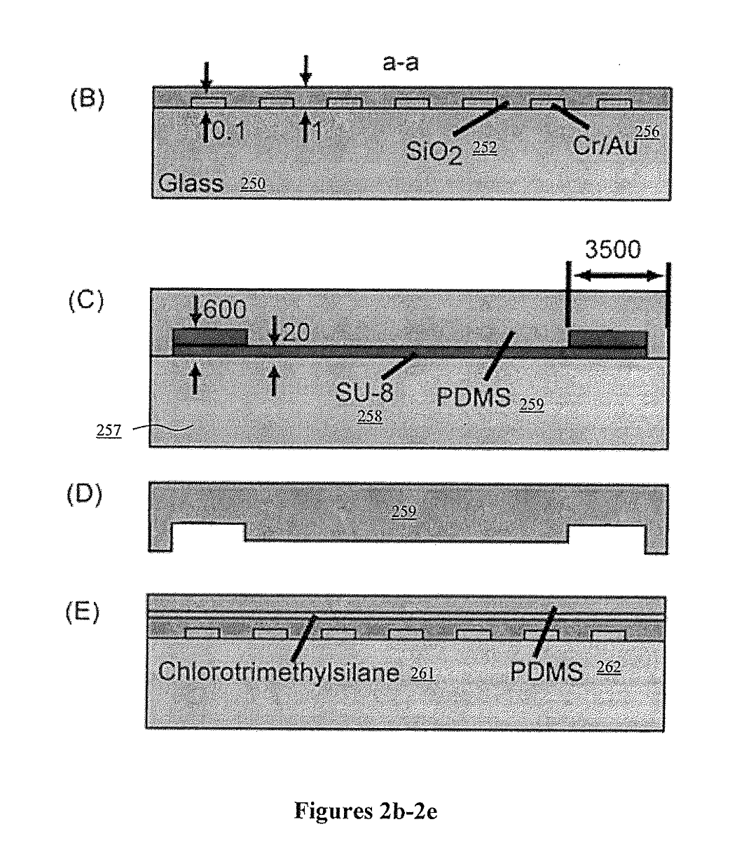

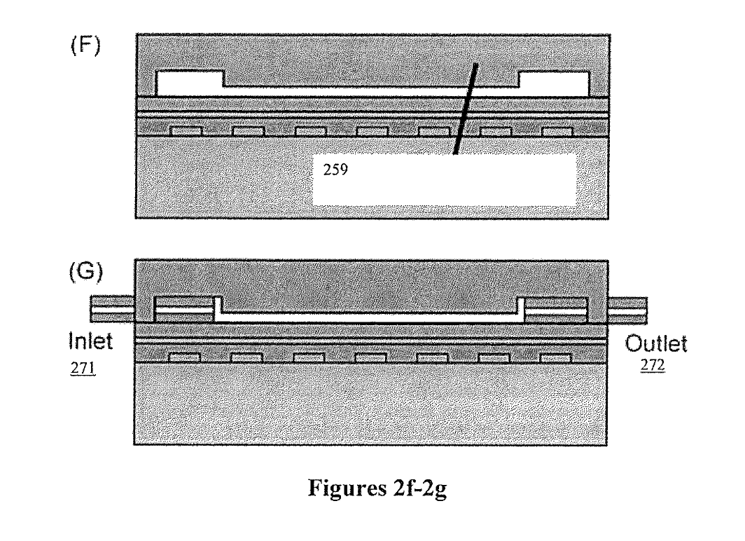

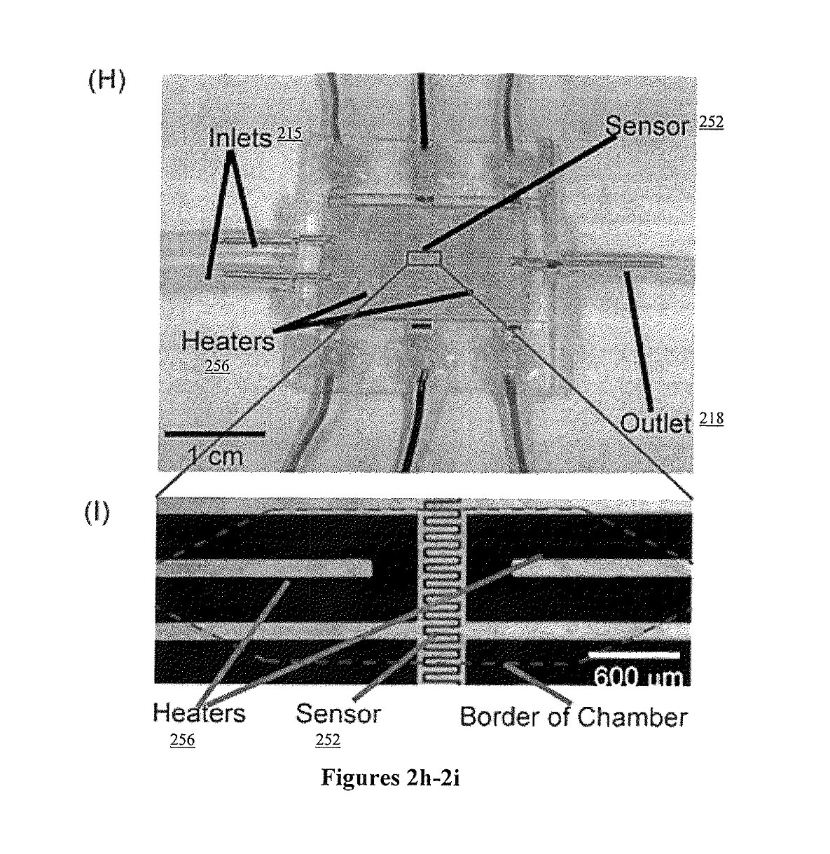

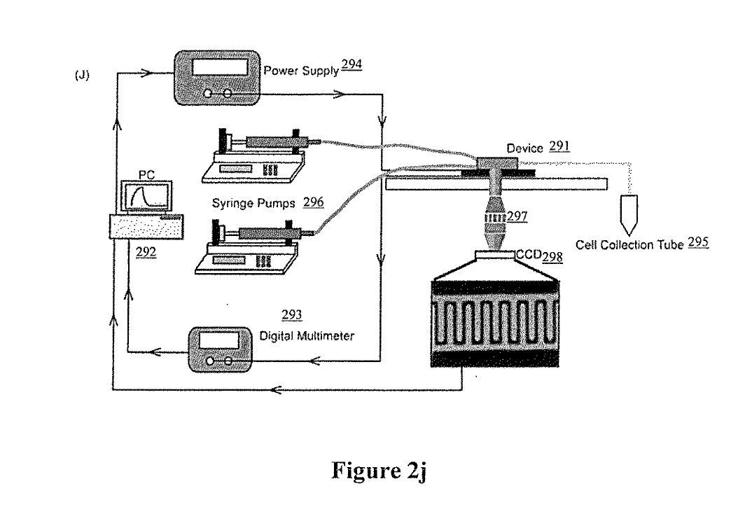

As illustrated in more detail in FIG. 2a, the microfluidic device used for cell capture and temperature-mediated cell release includes a microchamber 210 situated on a temperature control chip 230. The tapered chamber (2 mm in length, 1 mm in width and 20 μm in height), whose surfaces are functionalized with aptamers specific to a target cell type, is connected to two inlets 215 (3.5 mm in length, 0.7 mm in width and 600 μm in height) respectively for introduction of sample and washing buffer, and an outlet 218 for collection of released cells or waste fluids. The microfluidic channels connecting these fluidic ports and the chamber are 0.5 mm in width and 20 μm in height. Integrated on the temperature control chip 230 are a serpentine-shaped temperature sensor 252 (linewidth: 25 μm) beneath the center of the chamber, and two serpentine-shaped heaters 256 (linewidth: 300 μm) on each side of the temperature sensor. The chamber temperature can be controlled in closed loop using these integrated temperature sensor and heaters.

The temperature control chip 230 was fabricated using standard microfabrication techniques. A glass slide (Fisher HealthCare, Houston, Tex.) was cleaned by piranha. Chrome (10 nm) and gold (100 nm) thin films 256 were deposited by thermal evaporation and patterned by wet etching to generate the temperature sensor and heaters which were then passivated by 1 μm of silicon dioxide that was deposited using plasma-enhanced chemical vapor deposition (PECVD). Finally, contact regions for electrical connections to the sensor and heaters were opened by etching the oxide layer using hydrofluoric acid (FIG. 2b).

Separately, the microchamber 210 was fabricated from polydimethylsiloxane 259 (PDMS) (Sylgard 184, Dow Corning Inc. Midland, Mich.) using soft lithography techniques. Layers of SU-8 photoresist 258 (MicroChem Corp., Newton, Mass.) were spin-coated on a silicon wafer 257 (Silicon Quest International, Inc., San Jose, Calif.), exposed to ultraviolet light through photomasks, baked, and developed to form a mold defining the microfluidic features. Next, a PDMS prepolymer solution (base and curing agent mixed in a 10:1 ratio) was cast onto the mold and cured on a hotplate at 72° C. for 1 hour (FIG. 2c). The resulting sheet bearing the microfluidic features was then peeled off the mold (FIG. 2d).

Subsequently, the surface of the temperature control chip was treated with chlorotrimethylsilane 261, and a PDMS layer 262 (approximately 100 μm) was spin-coated onto the chip (FIG. 2e). Then, the PDMS sheet 259 was bonded to the PDMS layer 262 after treatment of the bonding interfaces with oxygen plasma for 15 seconds (FIG. 2f). Finally, capillary tubes (O.D.=813 μm and I.D.=495 μm) were inserted into the inlet port 271 and outlet port 272 (FIG. 2g), resulting in a packaged device. Following each test, the PDMS sheet 250 can be easily removed from the temperature control chip, allowing the temperature control chip to be reused for the next test. A fabricated and packaged device is shown in FIG. 2h, and a close-up image of a selected portion of the device is shown in FIG. 2i.

The materials used in this Example were obtained as follows. Chlorotrimethylsilane, (3-mercaptopropyl)trimethoxysilane (3-MPTS), 4-maleimidobutyric acid Nhydroxysuccinimide ester (GMBS), streptavidin and bovine serum albumin (BSA) were obtained from Sigma-Aldrich (St. Louis, Mo.). 5,5′,6,6′-tetrachloro-1,1′,3,3′-tetraethylbenzimidazolylcarbocyanine iodide (JC-1), propidium iodide (PI), RPMI-1640 media, fetal bovine serum (FBS), penicillinstreptomycin (P/S, penicillin 10,000 unit/mL, streptomycin 10,000 μg/ml), Dulbecco's phosphatebuffered saline (D-PBS) and the Vybrant® multicolor cell-labeling kit (DiI, DiO and DiD) were purchased from Invitrogen (Carlsbad, Calif.). CCRF-CEM and Toledo cell lines were obtained from the American Type Culture Collection (ATCC, Manassas, Va.). The biotinylated sgc8c aptamer with a polyT(9) spacer at the 5′ end of the sequence (biotin-5′-TT TTT TTT TAT CTA ACT GCT GCG CCG CCG GGA AAA TAC TGT ACG GTT AGA-3′ (SEQ ID NO:2), Kd=0.78 nM) was synthesized and purified with high-performance liquid chromatography (HPLC) by Integrated DNA Technologies (Coralville, Iowa).

The biotinylated sgc8c aptamer was functionalized in a freshly fabricated microdevice. The microchamber was first treated with 4% (v/v) 3-MTPS in ethanol for 30 min at room temperature, followed by an ethanol wash. 2 mM GMBS in ethanol was then introduced and incubated for 20 min at room temperature, followed by an ethanol wash and drying by nitrogen. The chamber was incubated overnight with 100 μg/mL streptavidin in D-PBS at 4° C., followed by a D-PBS wash. Finally, 10 μM of biotinylated sgc8c aptamer in D-PBS was introduced into the chamber and incubated at room temperature for 20 min. A D-PBS wash was used to remove free aptamer molecules, leaving immobilized aptamer molecules on the surface. Prior to cell introduction, the chamber was incubated with 1 mg/mL BSA solution in D-PBS at room temperature for at least 30 min to minimize nonspecific adsorption of cells.

Both CCRF-CEM and Toledo cells were incubated with RPMI-1640 media supplemented with 10% FBS and 1% P/S, and were kept at 37° C. in a humidified incubator containing 5% CO₂. Each cell type was collected through centrifugation, resuspended at 1×10⁸ cells/mL in D-PBS supplemented with 1 mg/mL BSA, and then kept on ice. Cells were mixed or diluted to different concentrations prior to introduction into the microdevice.

An example setup for capture and release of target cells using the microdevice is shown in FIG. 2j. Closed-loop temperature control of the microchamber of the microdevice 291 was achieved using the integrated temperature sensor and heaters (not shown) with a proportional-integral-derivative (PID) algorithm implemented in a LabVIEW (National Instruments Corp., TX) program on a computer 292. The resistance of the sensor was measured by a digital multimeter (34420A, Agilent Technologies Inc., CA), and the heaters were connected to a DC power supply 294 (E3631, Agilent Technologies Inc., CA). The inlets of the microdevice were connected to two syringes that respectively contained cell mixture and D-PBS, and was each driven by a syringe pump 296 (KD210P, KD Scientific Inc., MA). The outlet was connected to a microcentrifuge tube 295 for collection of released cells or waste. Unless indicated otherwise, all phase contrast images and fluorescent images of the chamber were taken using an inverted epifluorescence microscope (Diaphot 300, Nikon Instruments Inc., NY) with a CCD camera (Model 190CU, Micrometrics, NH).

During cell capture, a batch of CCRF-CEM cells was introduced into the chamber and incubated without any fluid flow for 1 min. This was repeated several times, followed by a wash with D-PBS at 5 μL/min for approximately 1 min. An image of the cell-laden chamber was taken and used to manually count the number of captured cells, which was used to compute the captured cell density on the surface. To test the specificity of cell capture, CCRF-CEM and Toledo cells were labeled with the fluorescent dyes DiO and DiI, respectively, and fluorescent images were taken after the first introduction of the cell mixture as well as after D-PBS washing.

In temperature-mediated cell release, the chamber was heated using the integrated heaters via closed loop temperature control to a desired temperature for 2 min, and flows of D-PBS at various rates were used to rinse the chamber, images of the chamber were taken every 2 seconds, and used to manually count the cells that remained on the aptamer-immobilized surface.

To test cell viability, the retrieved cells were kept in D-PBS with 10% FBS containing PI (2 μM) and JC-1 (10 μg/mL) at 37° C. for 1 hour, and then phase contrast and fluorescent images were taken with an inverted microscope (DMI6000B, Leica Microsystems Inc., IL) equipped with a digital camera (Retiga 2000R, Qimaging, Canada) and commercial image acquisition software (InVitro, Media Cybernetics Inc., MD). Moreover, a batch of cells was treated in a water bath at 48° C. for 2 minutes and then cultured for 4 days. The concentration of cultured cells was determined each day using a hemacytometer (Chang Bioscience Inc., CA).

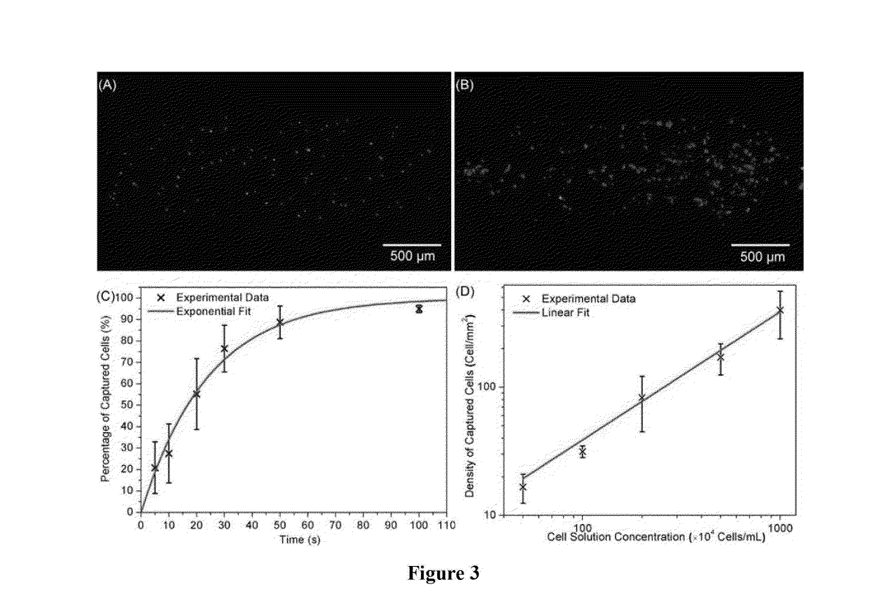

To verify specific cell capture at room temperature, a mixture of CCRF-CEM cells (target cell type, 3.5×10⁶ cells/mL) and Toledo cells (non-target cell type, 5.0×10⁶ cells/mL) was introduced into the sgc8c aptamer-modified microchamber and incubated for 1 min. As shown in FIG. 3a, the total number of CCRF-CEM cells observed in the microchamber, 51 in total, was less than that of Toledo cells, 78 in total. However after washing, all non-specifically adsorbed Toledo cells were removed, leaving only specifically captured CCRF-CEM cells. Moreover, after 10 cell samples were introduced (each followed by rinsing with D-PBS), the target cells dominated the chamber surface, with only 8 non-target Toledo cells visible amongst a few hundred CCRF-CEM cells (FIG. 3b). This demonstrates the specific and effective capture of CCRF-CEM cells using the surface-immobilized aptamers, and the capability of the device to enrich target cells from a heterogeneous mixture.

To test the transient behavior of the cell capture process, CCRF-CEM cell suspensions with concentrations of 5.0×10⁶ cells/mL were introduced into the aptamer-functionalized chamber and allowed to incubate for varying lengths of time. After incubation, D-PBS was used to remove unbound cells. The fraction of captured cells in each introduction was calculated by η≈Nₐ/N_{b}, where Nₐ is the number of captured cells, i.e., cells that remained on the microfluidic aptamer-functionalized chamber surface after washing, and N_{b} is the maximum number of cells that can be captured due to geometric limitations. Because of the height of the chamber (20 μm) and the low cell density of the introduced cell suspension, it was assumed that only a single monolayer of cells could be arranged on the lower surface of the chamber. Under this assumption, N_{b} is also equal to the number of cells observed in the chamber before washing.

As shown in FIG. 3c, increasing incubation time resulted in an increase in cell surface density. The captured cell percentage (calculated from three repeated tests, n=3) revealed an approximately exponential dependence on incubation duration η=1−e^{-t/τ}, where τ is a constant, and t is the incubation duration. According to this relationship, cell loss during washing could be eliminated via incubation by setting t (incubation time) to a value such that η approximates 1. The constant τ indicates the rate at which the surface concentration of captured cells approaches its maximum value, and can be used to calculate the time needed to isolate a number of target cells from the heterogeneous cell suspension. An exponential fit to the test data indicated such a relationship (coefficient of determination R²=0.982), and yielded a value of τ equal to 24 s. Based on this first-order exponential fit, it was estimated that approximately 92% of introduced cells exposed to the aptamer-functionalized surface were captured after incubating for 1 min. These results, which were similarly obtained at other cell concentrations ranging from 0.5×10⁶ to 10×10⁶ cells/mL, can be further improved by selecting appropriate chamber design, surface topography, and operation conditions such as flow rates.

The effects of the cell suspension concentration on the surface density of captured cells were also determined. Cell capture was conducted using samples with varying cell concentrations (0.5 to 10×10⁶ cells/mL). In each test, 5 aliquots of cells were introduced into the chamber, each followed by a 1-min incubation. Each test was performed in triplicate simultaneously on identical devices (n=3). All of the devices were fabricated at the same time to guarantee chamber surfaces were generated with nominally identical aptamer densities to ensure consistent test data. Tests with the most dilute cell suspension (0.5×10⁶ cells/mL) yielded captured cells with a surface density of 17±4 cells/mm² (n=3), while those with the most concentrated cell suspension (10×10⁶ cells/mL) resulted in a captured cell density of approximately 399±160 cells/mm² (n=3), as shown in FIG. 3d. It can be seen that in this range of cell concentrations, the captured cell density was approximately proportional to the cell concentration ρ_{capture}=A c_{cell}, where c_{cell} is the cell suspension concentration (cells/mL), and A is a proportionality constant that depends on device characteristics such as the surface density of immobilized aptamer molecules and equilibrium cell-aptamer affinity association, and testing parameters such as the number of samples introduced to the chamber. The linear equation fitted the test data (R²>0.99), resulting in a value of A equal to 0.3874 mL/mm². These results indicate that there is a large dynamic range of cell suspension concentrations over which the device can capture cells with good predictability for downstream analysis.

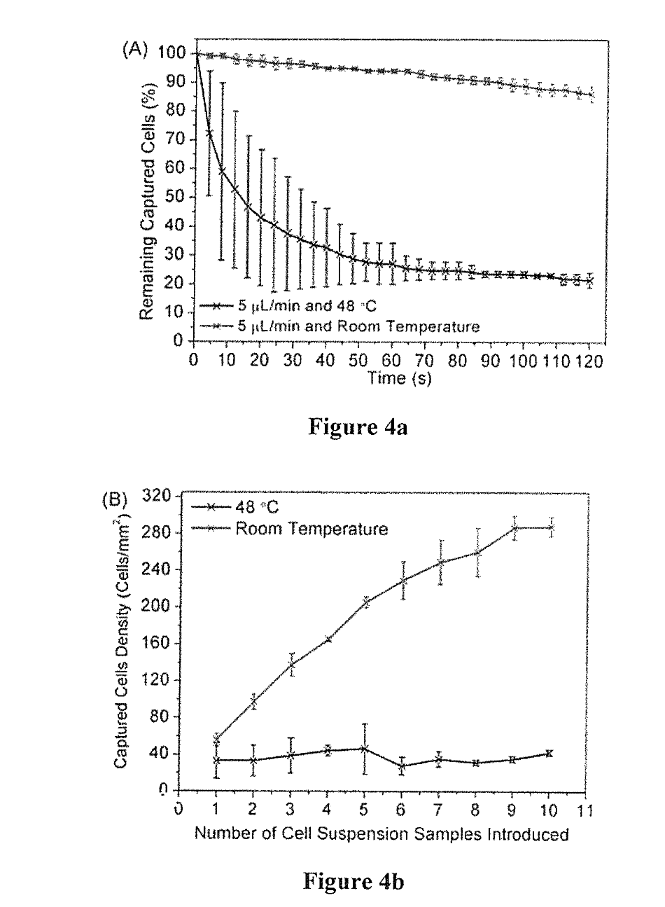

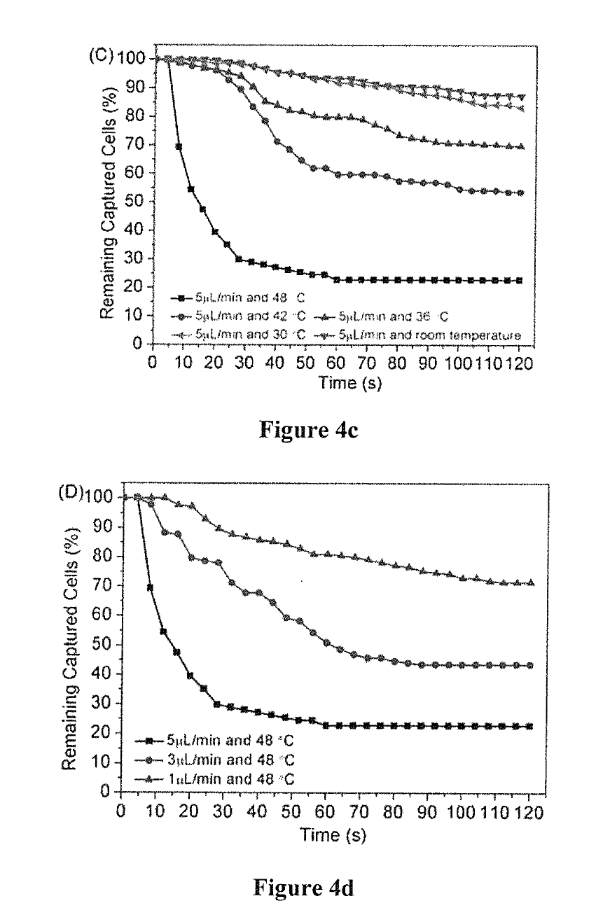

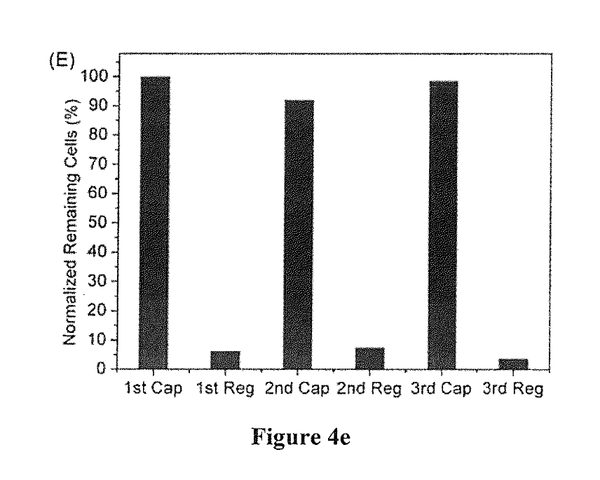

The thermally induced release of captured cells from the aptamer-functionalized chamber surfaces were further tested. Prior to the test, CCRF-CEM cells were captured by the surface-immobilized sgc8c aptamer, and unspecific bound cells were removed by D-PBS washing. Then, the cell-laden chamber was rinsed at either room temperature or 48° C. (FIG. 4a). Approximately 80% of cells were released from the surfaces after rinsing with D-PBS at 5 μL/min and 48° C. for 2 min, whereas negligible cell release was observed when rinsing at room temperature with an identical buffer solution and flow rate. These results suggest that the release of CCRF-CEM cells can be caused by the conformational changes in the aptamer structure at the elevated temperature.

Additional tests were conducted in which cells were heated prior to capture in the device, and compared the results to those from heating the device itself during cell capture. The cell suspension, diluted to 5×10⁶ cells/mL, was heated at 48° C. for 2 min, followed by introduction to the chamber at room temperature. In parallel, an unheated cell solution of 5×10⁶ cells/mL was introduced into a chamber with the chamber temperature set to 48° C. In both tests, 10 aliquots of cells were introduced into the chamber, followed by 1 min of incubation after each cell introduction. Heat treated cells were captured at room temperature up to a concentration of 288±10 cells/mm² (n=3), as shown in FIG. 4b. Unheated cells in a 48° C. chamber achieved a surface density of only 43±3 cells/mm² (n=3), and the presence of these remaining surface-bound cells was attributed to non-specific adsorption. These results show that the conformational changes in the aptamer structure, rather than the denaturation of the target cell membrane protein PTK7 at the increased temperature, caused the release of the specifically captured cells.

The impact on cell release by the chamber temperature was compared to the hydrodynamic shear stress applied by the buffer flow. Cell detachment from aptamer-functionalized substrates is governed by the balance between the hydrodynamic shear stress applied on cell surfaces and the temperature-dependent binding strength of aptamers and their target cells. Therefore, changes in either the chamber temperature or the buffer flow rate can result in different cell release efficiencies. Thus, the effects of temperature on cell release were tested by varying the chamber temperature from 30° C. to 48° C. while rinsing with D-PBS (FIG. 4c). It can be seen that with the elevated temperature, an increasing number of cells were detached from the substrate. Moreover, as the local temperature increased from 30 to 48° C., the viscosity of the aqueous washing buffer can decrease by approximately 35%, which lead to about 35% lower shear stress at the cell membranes. This indicates that at higher temperatures there is a greater loss of binding between the aptamers and the cells, which can be due to temperature-dependent changes in conformational structure of aptamers.

The effect of shear stress on cell release was tested by performing similar tests while varying the flow rate through the chamber. As shown in FIG. 4d, a higher flow rate caused more cells to detach from the substrate, as a result of increased shear stress disrupting the cell-aptamer binding. As either a higher temperature or a larger shear stress poses a greater risk of cell damage, the tradeoff between them can be an important design consideration.

As conformational changes in aptamer structures are reversible, the cell-capture surface can be regenerated after the release of the captured cells. To verify the reusability of the aptamer-functionalized surface, three cycles were performed in the same device, with each cycle including first introducing a dilute cell solution to the microchamber at room temperature, then releasing cells at 48° C. and 5 μL/min for 2 min, and finally regenerating the aptamer-functionalized surface (releasing all remaining cells) via washing with D-PBS at 60° C. and 50 μL/min for 2 min, and then at room temperature and 50 μL/min for 2 min. Following the first cycle, similar densities of captured cells were observed for subsequent cycles, with a maximum difference of captured cell density of only 8% between the first and the second capture (FIG. 4e). These results indicate that the regeneration of cell capture function of the microfluidic device can be both effective and consistent. Although some residual cells remained on the surfaces after each regeneration, this can be addressed by using a higher temperature and flow rate.

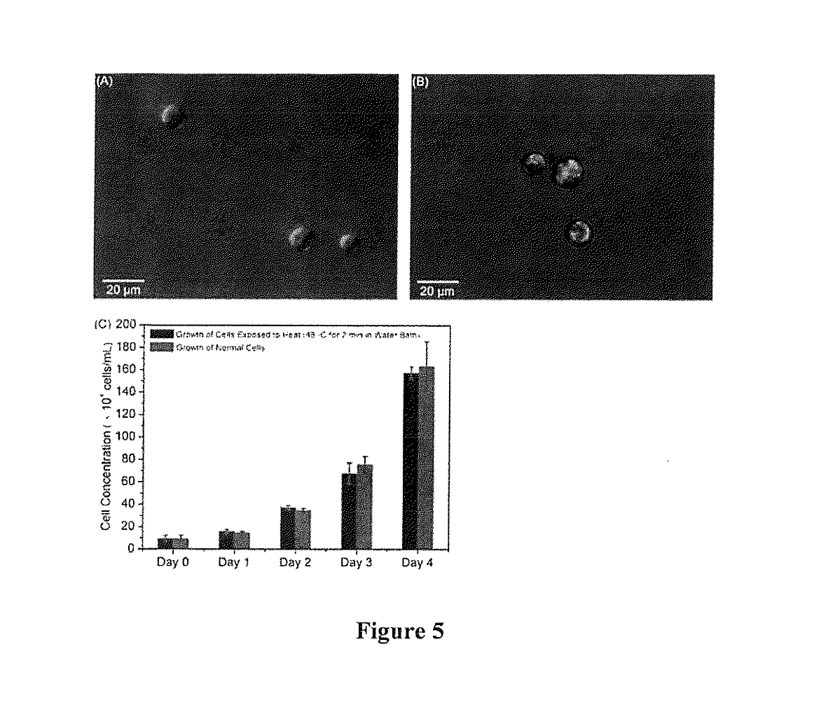

Cell viability is important for downstream applications such as tissue engineering and cell-based therapeutics. To evaluate cell viability, released cells were collected after rinsing at 5 μL/min and 48° C. for 2 min, at which point PI and JC-1 were used to stain cells. PI is a red-fluorescent nuclear stain that is not permanent to live cells. JC-1 accumulates in healthy mitochondria as indicated by red fluorescence, the intensity of which decreases along with mitochondrial depolarization occurring in the early stage of apoptosis. The results showed that the PI stained cells did not emit any red fluorescence (FIG. 5a), and the JC-1 stained cells exhibited bright red fluorescence (FIG. 5b), indicating that the collected cells were viable.

Cell viability was further confirmed by cell culture test. Off-chip cell proliferation assays were performed, in which cells from a well-mixed suspension were treated in water bath at 48° C. for 2 min and then cultured for several days. Meanwhile, cells from the same suspension were also cultured without any treatment for the same period to serve as a control. The growth curves of normal and heat-treated cells are shown in FIG. 5c, in which heat-treated cells are seen to have a similar proliferation rate as normal cells. This indicates that the brief period of modestly elevated temperature used in the cell release would not induce detectable cell damage, allowing the thermally released cells to remain viable.

### Example 2

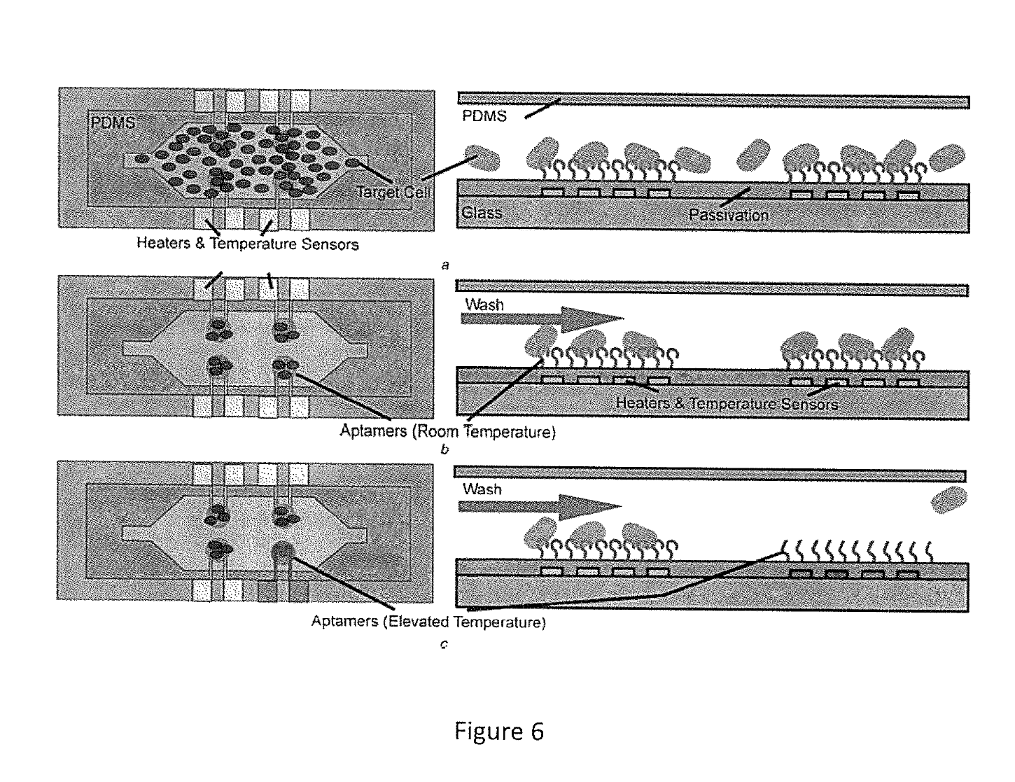

The principle of aptamer-based specific cell capture and spatially selective temperature-mediated cell release is as follows. Cell specific aptamers are first patterned on design-specified regions of the surface of a temperature-control chip. A cell suspension containing target cells is introduced into the device. Target cells located on the aptamer modified regions are captured specifically by the patterned aptamers (FIG. 6a), whereas those situated outside the aptamer-functionalized surface are not captured and removed by a Dulbecco's phosphate-buffered saline (D-PBS) wash (FIG. 6b). Next, the temperature of a specific region is increased to change the conformational structure of aptamers, by activating the microheater. Thus, the binding strength between target cells and aptamers is decreased. Cells within this region can then be washed away and collected, whereas cells in other regions are not affected (FIG. 6c). After the temperature is reversed, aptamers recover their ability to capture cells. In addition, this moderate temperature change does not affect cell viability. For a demonstration, the microfluidic device is functionalized with the aptamer sgc8c for specific capture and temperature-mediated release of CCRF-CEM cells, a human acute lymphocytic leukemia cell line.

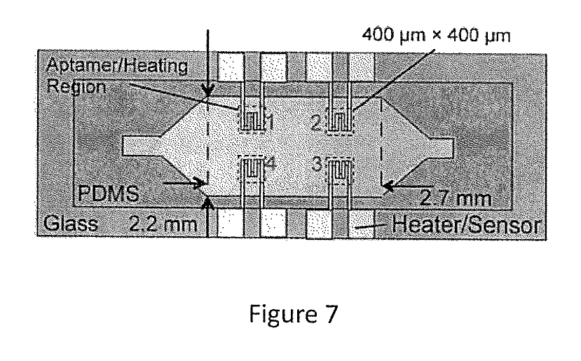

The microfluidic device used for specific cell capture and spatially selective temperature-mediated cell release can consist of a tapered microchamber (2.7 mm in length, 2.2 mm in width and 20 μm in height) situated on a microchip with four groups of serpentine-shaped heaters (linewidth: 50 μm) and serpentine-shaped temperature sensors (linewidth: 20 μm) (FIG. 7).

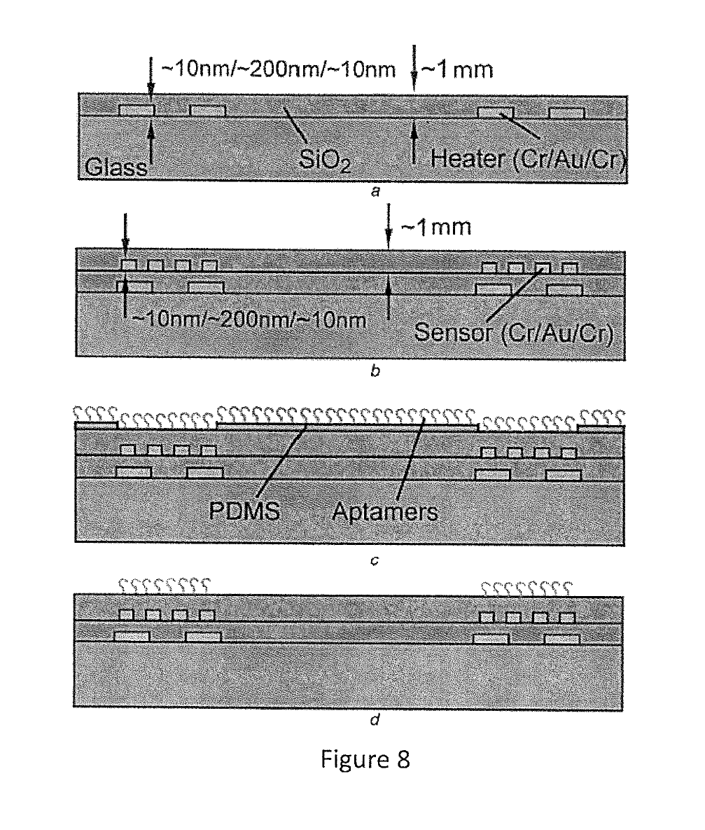

The microchip can be fabricated using standard microfabricatation techniques. Briefly, a chrome (˜10 nm)/gold (˜200 nm)/chrome (˜10 nm) thin film was first deposited and patterned to form microresistive heaters, which were then passivated by approximately 1 μm silicon dioxide using plasma-enhanced chemical vapour deposition (FIG. 8a). The microheaters generated joule heat when subjected to a DC voltage. Next, an additional chrome (˜10 nm)/gold (˜200 nm)/chrome (˜10 nm) thin film was deposited and patterned to form the temperature sensors, which were also passivated by approximately 1 μm silicon dioxide (FIG. 8b). Subsequently, the microchip was incubated with 4% (v/v) 3-mercaptopropyl trimethoxysilane (3-MPTS) in ethanol for 30 min at room temperature, followed by an ethanol wash. The microchip was then treated with 2 mM 4-maleimidobutyric acid N-hydroxysuccinimide ester (GMBS) in ethanol for 20 min at room temperature, followed by another ethanol wash and drying by nitrogen.

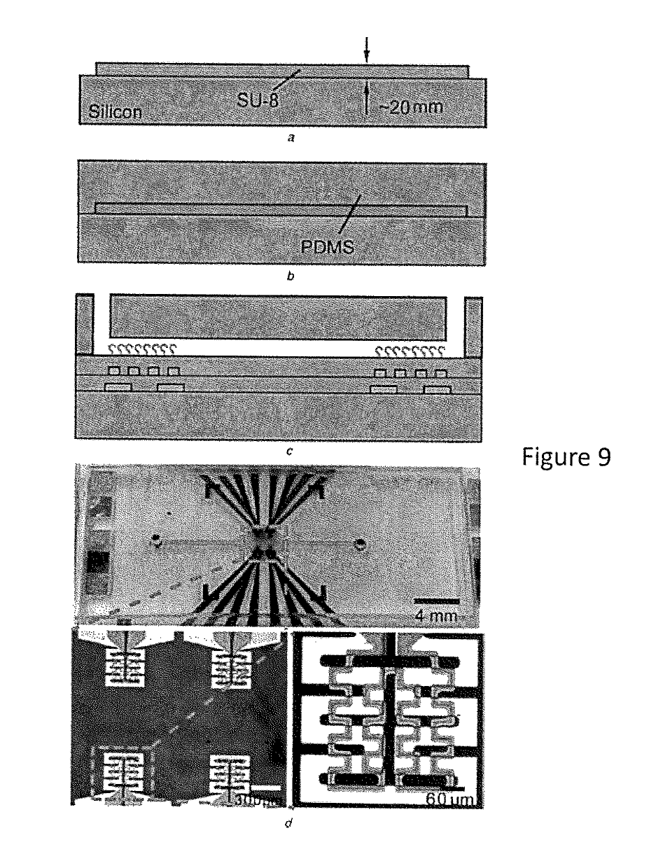

Afterwards, the microchip was incubated with 100 μg/ml streptavidin in D-PBS at 4° C. overnight, and a polydimethylsiloxane (PDMS) (Sylgard 184, Dow Corning Inc. Midland, Mich.) membrane with through openings (diameter: 400 μm) was manually attached onto the microchip surface, to which the biotinylated sgc8c aptamers were immobilized through biotin-streptavidin interaction. After peeling off the PDMS membrane, only aptamers immobilized on the microchip remained, and those modified on the membrane were removed (FIG. 8d). Finally, the microchamber was fabricated from PDMS using standard soft lithography methods (FIGS. 9a and b), and then attached onto the microchip (FIG. 9c). A fabricated and packaged microfluidic device is shown in FIG. 9d.

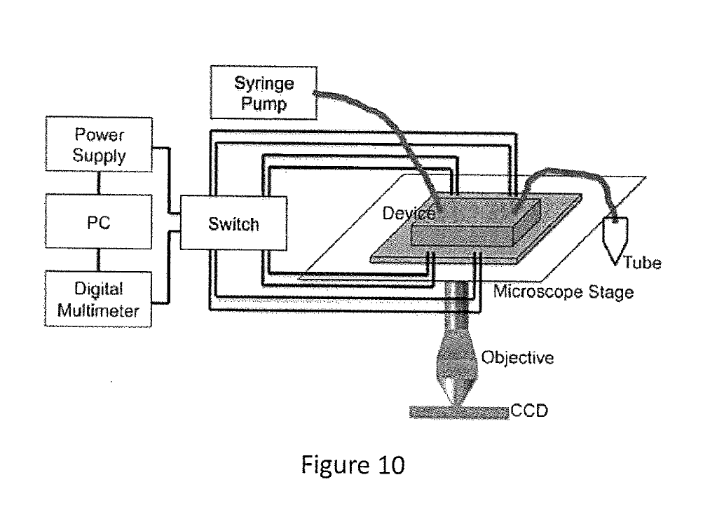

Closed-loop temperature control of each aptamer-modified region can be achieved by using the corresponding integrated temperature sensor and heater with a proportional-integral-derivative algorithm implemented in a LabVIEW (National Instruments Corp., TX) program on a personal computer. The sensor resistances can be measured by a digital multimeter (34 420 A, Agilent Technologies Inc., CA) through a 4-way mechanical switch. The microheaters can be connected to a DC power supply (E3631, Agilent Technologies Inc., CA) through another 4-way mechanical switch. The microfluidic device's inlet can be connected to a syringe driven by a syringe pump (KD210P, KD Scientific Inc., MA). The outlet can be connected to a microcentrifuge tube in order to collect released cells. Phase contrast images of cells captured on the microchip surface can be taken using an inverted epifluorescence microscope (Diaphot 300, Nikon Instruments Inc., NY) with a CCD camera (Model 190CU, Micrometrics, NH) (FIG. 10).

CCRF-CEM cells can be incubated with complete culture media that consisted of RPMI-1640 media supplemented with 10% FBS and 1% P/S, and kept at 37° C. in a humidified incubator containing 5% CO2. The cells can be collected through centrifugation, resuspended at 1×108 cells/ml in complete culture media with 1 mg/ml BSA and kept on ice. The microfluidic device can be first treated with 1 mg/ml BSA in D-PBS for at least half an hour. Then, a suspension of CCRF-CEM cells can be introduced into the microchamber at 1 μl/min for 2 min, followed by a D-PBS wash at 5 μl/min. An image of the microchip surface shows the specific capture of cells onto the aptamer-modified surface.

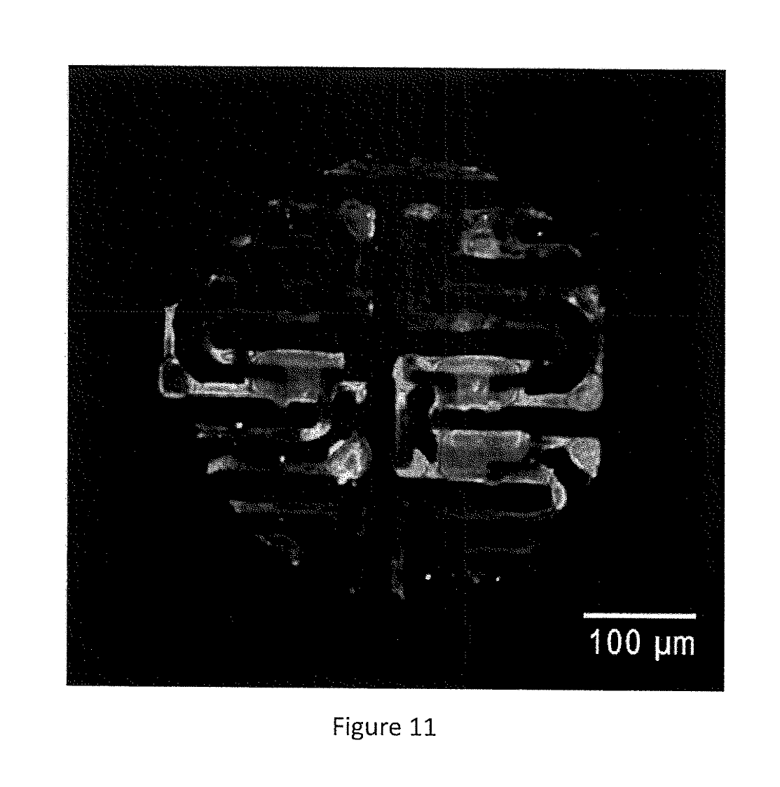

In the spatially selective temperature-mediated cell release experiments, the microchamber can be rinsed with complete culture media with 10 μg/ml JC-1 at 5 μl/min, and a selected region on the microchip can be heated using the integrated heater via closed-loop temperature control for 20 s. To test cell viability, the retrieved cells in complete culture media with 10 μg/ml JC-1 can be kept at 37° C. in an incubator with 5% CO2 for 1 h, and a fluorescent image taken with an inverted microscope (IX81, Olympus Corp., PA) equipped with a digital camera (C8484, Hamamatsu Corp., NJ). Fluorescently labelled biotinylated ssDNA can be used to functionalize the microchip, which can then be observed under a fluorescent microscope. As shown in FIG. 11, only the area exposed to reagents, which was in the through opening region, show bright green fluorescence, indicating the feasibility of immobilizing aptamers onto design-specified regions of a microchip.

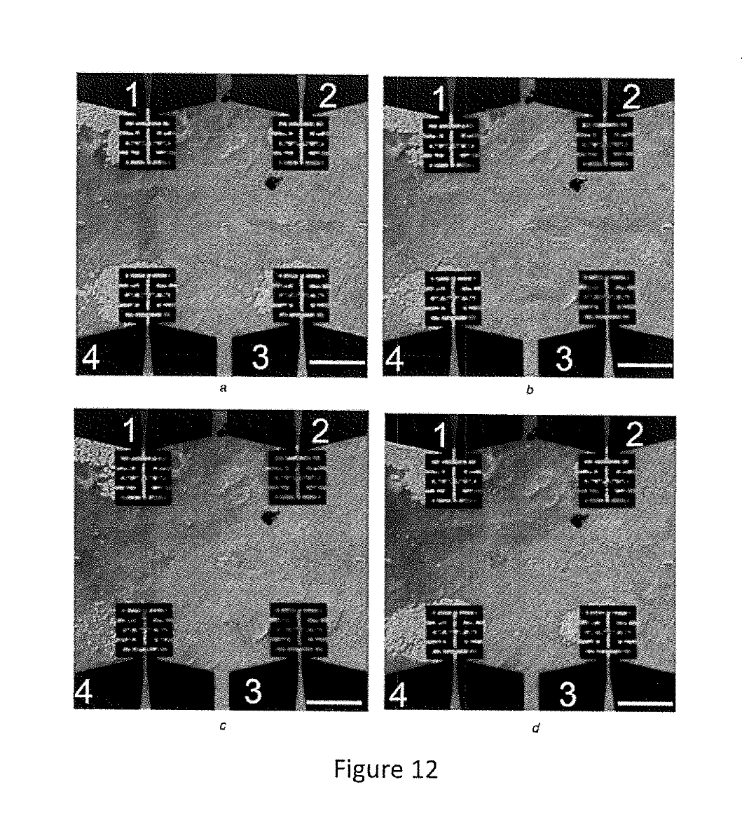

To demonstrate spatially selective cell capture, a CCRF-CEM cell suspension of 5×106 cell/ml with 1 mg/ml BSA can be introduced into the devices with immobilized aptamers at 1 μl/min for 2 min, followed by a D-PBS wash at 5 μl/min for 1 min. CCRF-CEM cells only became attached to the aptamer functionalized surfaces (FIG. 12a), and not to the bare surface, confirming spatially selective cell capture. Owing to the manually performed surface modification process, aptamers are not necessarily immobilized onto the surface above the microheaters. Therefore the shape of aptamer-captured CCRF-CEM cell patterns do not necessarily strictly follow the envelope of the microheaters.

The cell laden chamber can be rinsed with complete culture media with 10 μg/ml JC-1 at 5 μl/min, while the temperature in regions 2 and 3 increased to 48° C. in series, by using the integrated heaters. It can be seen that only the cells within regions 2 and 3 became detached from the aptamer-surface, which can be caused by conformational changes of the aptamer structure, whereas negligible cell release can be observed in other regions (FIG. 12b). The temperature in region 4 can be further increased, and noticeable cell release can be observed in this region, while cells in region 1 are not affected (FIG. 12c). These results indicate the success of temperature-mediated release of selected groups of cells.

To verify the reusability of the aptameric surface, another CCRF-CEM cell suspension with the same concentration can be introduced into the same device at 1 μl/min for 2 min. Following a D-PBS wash at 5 μl/min for 1 min, similar densities of captured cells can be observed in all the regions (FIGS. 12a and d), indicating that the microfluidic device with aptamers is reusable.

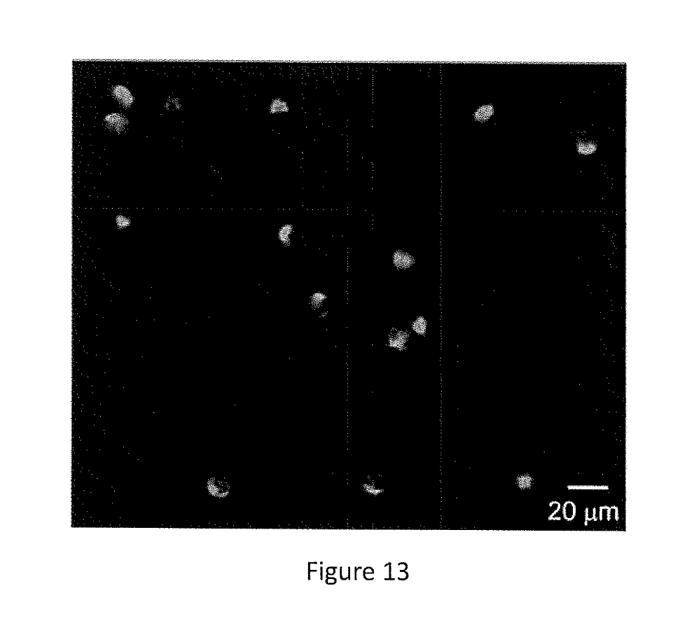

To enable downstream (e.g., tissue engineering and cell-based therapeutic) applications, the released and retrieved cells must be viable. To evaluate cell viability, released cells in complete culture media with 10 μg/ml JC-1 from multiple devices can be collected and incubated at 37° C. with 5% CO2 for 1 h, centrifuged and resuspended in 10 μl of complete culture media. JC-1 exists as a monomer in cytoplasma exhibiting green fluorescence and it accumulates in underpolarized healthy mitochondria showing red fluorescence, whose intensity decreases along with mitochondrial depolarization during apoptosis or death of cells. The released cells show bright red fluorescence (FIG. 13), indicating they are still viable and the temperature-mediated cell release process did not affect cell viability. In addition, to further decrease the potential cell damage, releasing cells at lower temperature is possible using appropriately selected aptamers.

1. A method for selectively capturing and releasing target cells using a microchamber including one or more microheaters, one or more temperature sensors, and one or more aptamers capable of binding with the target cells, comprising:
spatially arranging the one or more aptamers to create a surface pattern;
configuring the one or more microheaters and the one or more temperature sensors to align with the surface pattern;
introducing a sample including one or more target cells to the microchamber, to thereby bind the one or more target cells to the one or more aptamers at a first temperature of the microchamber; and
selectively using the one or more microheaters and the one or more temperature sensors to change the first temperature of at least one region of the microchamber to a second temperature to release one or more bound target cells from the aptamer at that region.

2. The method of claim 1, wherein the sample further includes one or more non-target cells, the method further comprising: washing the microchamber to remove cells not bound to the aptamer.

3. The method of claim 2, further comprising: after washing, introducing more sample including at least one additional target cell into the microchamber.

4. The method of claim 3, further comprising restoring the first temperature of at least one region, to thereby bind the at least one additional target cell in said region.

5. The method of claim 1, wherein the at least one target cell comprises a membrane protein, and wherein the target cell binds with the aptamer via the membrane protein.

6. The method of claim 5, wherein the membrane protein comprises one of PTK7, MUC1, PDGF, and PSMA or other proteins.

7. The method of claim 5, wherein the aptamer comprises an aptamer selected to specifically bind with the membrane protein.

8. The method of claim 1, further comprising: collecting and detecting the target cells.

9. The method of claim 1, further comprising immobilizing the aptamer on an inner surface of the microchamber.

10. The method of claim 1, wherein the first temperature comprises a temperature between 20 to 30° C.

11. The method of claim 1, wherein the first temperature comprises a temperature at about 37° C.

12. The method of claim 1, wherein the second temperature comprises a temperature between about 30° C. to about 55° C.

13. The method of claim 1, wherein the second temperature comprises a temperature between 4° C. to about 37° C.

14. A microdevice for selectively capturing and releasing target cells, comprising:
a microchamber including one or more spatially arranged aptamers capable of binding with the target cells; and
one or more temperature control elements aligned with said one or more aptamers and configured to regulate temperature of said aptamers.

15. The microdevice of claim 14, wherein the aptamer is immobilized on an inner surface of the microchamber.

16. The microdevice of claim 15, wherein the aptamer comprises a biotinylated aptamer which is immobilized on the inner surface of the microchamber via streptavidin-biotin binding or other attachment methods.

17. The microdevice of claim 14, wherein the aptamer comprises one of sgc8c, MUC1-5TR-1, xPSM-A9 and PDGF-aptamer-36t or another aptamer specific to the appropriate membrane protein.

18. The microdevice of claim 14, wherein the microchamber has a depth of between about 10 to about 100 μm.

19. The microdevice of claim 14, wherein the temperature control element comprises a resistive heater.

20. The microdevice of claim 14, wherein the temperature control element comprises an element for thermoelectric heating or cooling.

21. The microdevice of claim 14, further including a temperature sensor.
